(19) United States
(12) Patent Application Publication
(10) Pub. No.: US 2024/0102002 A1
(43) Pub. Date: Mar. 28, 2024

(54) SYSTEMS AND METHODS FOR EVALUATING DRUG COMBINATIONS

(71) Applicant: Hunter BioDiscovery, Inc., BOSTON, MA (US)

(72) Inventors: Tal Raz, Brookline, MA (US); Xiao Wang, Waltham, MA (US); Boryana Zhelyazkova PURSLEY, Dedham, MA (US); David Allan Weitz, Bolton, MA (US)

(73) Assignee: Hunter BioDiscovery, Inc., BOSTON, MA (US)

(21) Appl. No.: 18/276,116

(22) PCT Filed: Feb. 1, 2022
(86) PCT No.: PCT/IB2022/050838

§ 371 Date: Aug. 7, 2023

Related U.S. Application Data
(60) Provisional application No. 63/148,866, filed on Feb. 12, 2021.

(51) Int. Cl. C12N 15/10; C12Q 1/02

(52) U.S. Cl. CPC C12N 15/1065; C12Q 1/025

(57) ABSTRACT

A system includes a plurality of drug libraries, at least one comprising multiple droplets of multiple drugs, each drug associated with a corresponding unique drug identifier in the system, and a live cell library comprising multiple live cells or live cell lines, each live cell associated with a corresponding unique cell or cell line identifier in the system. A plurality of junctions combining drug droplets from the drug libraries and a live cell from the live cell library. At least one of said drug libraries produces a random stream of heterogenous drug droplets, the drug droplets corresponding to drugs in said drug libraries.

### COPYRIGHT STATEMENT

This patent document contains material subject to copyright protection. The copyright owner has no objection to the reproduction of this patent document or any related materials in the files of the United States Patent and Trademark Office but otherwise reserves all copyrights whatsoever.

### RELATED APPLICATIONS

This application is related to and claims the benefit of U.S. provisional patent application No. 63/148,866, filed Feb. 12, 2021, the entire contents of which are hereby fully incorporated herein by reference for all purposes.

### FIELD OF THE INVENTION

Aspects of this invention relate to evaluation of drug combinations, and, more particularly, to a microfluidic droplet platform for evaluating of drug combinations against live cells and methods and systems using the same.

### BACKGROUND

In treatment of many diseases, combinations of drugs often succeed where single therapies fail.

“[C]ombining anti-cancer drugs . . . with distinct mechanisms of action is the approach most likely to overcome single-agent resistance and produce sustained clinical remissions” NCI, Cancer Res; 77(13); 2017. “[I]mproved outcomes might be gained from the combination of antiviral therapy with drugs that modulate the immune response in an infected individual” THE LANCET, VOLUME 14, ISSUE 12, P1259-1270, 2014.

“When used appropriately, potent combinations of antiviral drugs seem to be able to circumvent the inherent tendency of HIV-1 to generate drug-resistant viruses” THE LANCET, HIV, VOLUME 348, ISSUE 9022, P239-246, 1996

“[E]vidence is accumulating that development of resistance will eventually limit the efficacy of new drugs. Thus, combinations of multiple agents will be required to treat chronic HCV infection”—NATURE. VOLUME 436, ISSUE 7053, P953-60, 2005

There is empirical evidence that combining drugs can interfere with biological pathways and provide therapeutic benefit. In the domain of cancer, combination therapies are largely adopted. However, they are often identified experimentally by combining new investigational drugs with existing first line therapies for additive effects in a clinical setting. To the best of our knowledge, there has not been a systematic evaluation of drug combinations in cellular assays and pre-clinical studies. An economical and effective way to evaluate drug combinations, could highlight new and unexplored therapeutic approaches of clinical benefit. Unfortunately, current screening technologies rely on automated liquid handling. This approach has significant limitations. Although the primary method leverages automated pipetting, it cannot achieve the ultra-high throughput required to test the vast number of combinations. So combination testing is slow. In addition, robotic liquid handling uses significant consumables and reagent volumes which is expensive. As a result, screening combinations have been limited to relatively small drug libraries, leaving the vast landscape of candidate combinations, largely unexplored.

The NCI published the landmark Cancer Almanac in 2017 screened 5,000 combinations and identified two pairs worthy of human clinical trials. This screen involved a laborious robotic process that cost the NCI˜$2 M and took 2.5 years.

It is desirable and an object hereof to identify novel drug combination therapies.

It is further desirable and a further object hereof to identify novel drug combination

therapies in a cost-effective and efficient manner.

It is further desirable and a further object hereof to provide mechanisms for identifying or screening potential novel drug combination therapies.

### SUMMARY

The present invention is specified in the claims as well as in the below description. Preferred embodiments are particularly specified in the dependent claims and the description of various embodiments.

One general aspect includes a plurality of drug libraries, at least one may include multiple droplets of multiple drugs, each drug associated with a corresponding unique drug identifier in the system. The system also includes a live cell library having multiple live cells, each live cell type associated with a corresponding unique cell identifier in the system. The system also includes a plurality of junctions combining drug droplets from the drug libraries and a live cell from the live cell library.

Implementations may include one or more of the following features, alone and/or in combination(s):
The system where the plurality of junctions form a plurality of merged sets, each of the merged sets may include a particular cell from the live cell library and a drug droplet from each of the plurality of drug libraries.
A merge set is uniquely identifiable by: (i) the unique cell identifier of the particular cell, and (ii) the unique drug identifiers of the drug droplets may include the merge set.
Drugs in a merge set are identifiable by the identifiers of the drug droplets may include the merge set.
The system further constructed and adapted to determine, from plurality of merged sets, effectiveness of a set or sets of drugs with respect to one or more criteria; and identify the drugs in the set or sets of drugs that were the effective with respect to the one or more criteria.
The system where the one or more criteria are selected from: live versus dead, protein abundance, presence or intensity of cellular proteins, metabolites or metabolite detection, nucleic acid detection, DNA, RNA, and/or other biomarkers.
The system identifies the set or sets of drugs that were most effective with respect to the one or more criteria.
The system identifies one or more drug combinations based on the drug combination's synergistic effect on a cell according to the one or more criteria.
The unique drug identifier for a drug in the plurality of drug libraries may include a unique DNA sequence for the drug, and where the unique cell identifier for a cell in the live cell library may include a unique DNA sequence for the cell, and where the system is constructed and adapted to identify the drugs in the set or sets of drugs by concatenating the unique DNA sequences in each set; and sequencing concatenated unique DNA sequences.
The plurality of junctions combine streams of drug droplets from each of the plurality of drug libraries with a stream of live cells from the live cell library.
The plurality of junctions may include: one or more first junctions for combining a stream of drug droplets from each of the plurality of drug libraries.
The one or more first junctions form a stream of drug droplet sets.
The plurality of junctions further may include a second junction combining live cells from the live cell library with the drug droplet sets.
The second junction forms a plurality of merged sets, each of the merged sets may include one or more of a particular cell type from the live cell library and a drug droplet from each of the plurality of drug libraries.
Each merge set is uniquely identifiable by the unique cell identifier of the particular cell type and the identifiers of the drug droplets.
The corresponding unique drug identifier for a drug in the drug libraries may include a unique DNA drug identifier.
The corresponding unique cell identifier for cell types in the live cell library may include a unique DNA cell identifier.
The unique DNA drug identifier for a drug in the plurality of drug libraries may include a unique DNA sequence for the drug.
The unique DNA cell identifier for a cell in the live cell library may include a unique DNA sequence for the cell.
Drugs in a merge set are identifiable by the unique DNA drug identifiers of the drug droplets may include the merge set.
The plurality of drug libraries may include of two drug libraries.
Each of the plurality drug libraries contains up to 1,000 drugs, more preferably up to 2,000 drugs, and even more preferably up to 5,000 drugs.
The live cell library contains 1 to 100 cell lines.
Each of the drug libraries produces a stream of drug droplets, the drug droplets corresponding to the drugs in the drug libraries.
At least one of said drug libraries produces a stream of heterogenous drug droplets, the drug droplets corresponding to drugs in drug libraries.
At least one of the drug libraries produces a random stream of heterogenous drug droplets.
The unique drug identifier for a drug in the system may include an optical identifier associated with the drug.
The optical identifier may include a dye.

Another general aspect includes a method that includes merging a set of drugs from a plurality of drug libraries with live cells from a live cell library to form corresponding merged sets, where at least some of the plurality of drug libraries may include multiple droplets of multiple drugs, each drug associated with a corresponding unique drug identifier, and where the live cell library may include multiple live cells, each live cell associated with a corresponding unique cell identifier. The method also includes determining, from the cells that responded, effectiveness of a set or sets of drugs with respect to one or more criteria. The method also includes identifying the drugs in the set or sets of drugs that were effective with respect to the one or more criteria.

Implementations may include one or more of the following features, alone and/or in combination(s):
The method where the identifying identifies the set or sets of drugs that were most effective with respect to the one or more criteria.
The one or more criteria are selected from: live versus dead, protein abundance, metabolite detection, nucleic acid detection.
The method may include incubating the merged sets prior to the determining.
The method may include, prior to the determining: partitioning the merged sets based on their cells' responses to the drugs, according to the one or more criteria.
The partitioning may include separating cells that responded to the drugs according to the one or more criteria from cells that did not respond according to the one or more criteria.
The method may include determining a matrix of drug combination responses.
An entry in the matrix for a particular drug combination corresponds to the particular drug combination's synergistic effect on a cell according to the one or more criteria.
The unique drug identifier for a particular drug may include a unique DNA sequence for the particular drug, and where the unique cell identifier for a particular live cell may include a unique DNA sequence for the particular live cell.
The identifying the drugs may include concatenating the unique DNA sequences in each set.
The method may include sequencing concatenated unique DNA sequences.
The unique drug identifier for a particular drug may include a unique optical identifier for the particular drug, and where the unique cell identifier for a particular cell may include a unique optical for the particular cell.
Each merge set is uniquely identifiable by the unique cell identifier of the cell in the merge set and the identifiers of the drugs in the merge set.
The method may include creating the plurality of drug libraries.
The method may include creating the live cell library.
The plurality of drug libraries may include of two drug libraries.
Each of the drug libraries contains up to 1,000 drugs, more preferably up to 2,000 drugs, and even more preferably up to 5,000 drugs.
Each of the drug libraries produces a stream of drug droplets, the drug droplets corresponding to the drugs of the drug libraries.

Another general aspect includes a system with a plurality of drug libraries, each may include multiple droplets of multiple drugs, each drug drop may also include or be associated with a corresponding unique DNA drug identifier. The system also may include a live cell library which may include multiple live cells, each live cell droplet may include a corresponding unique DNA cell identifier. The system also includes one or more first junctions for combining a stream of drug droplets from each of the plurality of drug libraries to form a stream of drug droplet sets. The system also includes a second junction for combining live cells from the live cell library with the drug droplet sets.

Implementations may include one or more of the following features, alone and/or in combinations:
The system where each drug is compartmentalized in aqueous droplets.
The system where the multiple live cells are in aqueous solution or cells compartmentalized in aqueous droplets.
The system where the plurality of drug libraries may include two drug libraries.
The system where each of the drug libraries contains up to 1,000 drugs, more preferably up to 2,000 drugs, and even more preferably up to 5,000 drugs.
The system where each of the drug libraries produces a stream of drug droplets.
The system where each of the drug libraries produces a stream of drug droplets, each droplet including a DNA drug-identifier.
The system where the unique DNA drug identifier for a drug in the plurality of drug libraries may include a DNA barcode for the drug, and where the unique DNA cell identifier for a cell in the live cell library may include a DNA barcode for the cell.
The system where the second junction forms a plurality of merged sets, each of the merged sets may include a particular cell from the live cell library and a drug droplet from each of the plurality of drug libraries.
The system where the second junction forms a plurality of merged sets, each of the merged sets may include a particular cell, and cell identifier from the live cell library and a drug and drug identifier droplet from each of the plurality of drug libraries.
The system where each merged set is uniquely identifiable by the DNA cell identifier of the particular cell in the set and the DNA identifiers of the drug droplets that formed the set.

Another general aspect includes a method that includes merging a set of drugs from a plurality of drug libraries with live cells from a live cell library to form corresponding merged sets, where each of the plurality of drug libraries may include multiple droplets of multiple drugs, each drug solution combined with a corresponding unique DNA drug identifier, and where the live cell library may include multiple live cells, each live cell solution with a corresponding unique DNA cell identifier. The method also includes incubating the merged sets; and then separating cells according to one or more criteria. The method also includes determining, from the cells that satisfied at least some of the one or more criteria, which set or sets of drugs were the most effective with respect to the one or more criteria. The method also includes identifying the drugs in the set or sets of drugs that were the most effective with respect to the one or more criteria.

Implementations may include one or more of the following features, alone and/or in combination(s):
The method where the one or more criteria comprise: cell death, presence or intensity of cellular proteins, metabolites, DNA, RNA, or other biomarkers.
The method includes determining a matrix of drug combination responses.
The method where a matrix entry in the matrix for a particular drug combination corresponds to the particular drug combination's synergistic effect on the cell, satisfying the one or more criteria.
The method where a merge set is uniquely identifiable by the DNA cell identifier of the cell in the merge set and the DNA identifiers of the drugs in the merge set.
The method where the method includes creating the plurality of drug libraries.
The method where the method includes creating the live cell library.
The method where the plurality of drug libraries consists of two drug libraries.
The method where each of the drug libraries contains up to 1,000 drugs, more preferably up to 2,000 drugs, and even more preferably up to 5,000 drugs.
The method where each of the drug libraries produces a stream of drug droplets, each with an associated drug DNA identifier.
The method where at least one of the drug libraries produces a heterogenous stream of drug droplets, each with an associated drug DNA identifier.
The method where at least one of the drug libraries produces a random stream of heterogenous drug droplets, each with an associated drug DNA identifier.

Another general aspect includes a system comprising a plurality of libraries; and one or more junctions combining droplets from the libraries, wherein each droplet is identifiable within the system.

Implementations may include one or more of the following features, alone and/or in combination(s):
The system where the one or more junctions form a plurality of merged sets of droplets, wherein a merged set has a droplet from each of the plurality of libraries.
The system where the droplets from at least one library comprise a stream of heterogenous drug droplets.
The system where the droplets from at least one library comprise a random stream of heterogenous drug droplets.
The system where the plurality of libraries comprise a plurality of drug libraries, at least one comprising multiple droplets of multiple drugs, each drug associated with a corresponding unique drug identifier in the system.
The system where the plurality of drug libraries consists of two drug libraries.
The system where the plurality of libraries comprise a live cell library comprising multiple live cells or live cell lines, each live cell associated with a corresponding unique cell or cell-line identifier in the system.

Another general aspect includes a method, in a system comprising a plurality of libraries; and one or more junctions, the method comprising: combining droplets from the libraries, wherein each droplet is identifiable within the system.

Below is a list of system embodiments. Those will be indicated with a letter “S”. Whenever such embodiments are referred to, this will be done by referring to “S” embodiments.

S1. A system comprising:
a plurality of drug libraries, at least one comprising multiple droplets of multiple drugs, each drug associated with a corresponding unique drug identifier in the system;
a live cell library comprising multiple live cells, or live cell-lines, each live cell associated with a corresponding unique cell or cell-line identifier in the system; and
a plurality of junctions combining drug droplets from the drug libraries and a live cell from the live cell library.

S2. The system of the system embodiment S1, wherein the plurality of junctions form a plurality of merged sets, each of said merged sets comprising a particular cell from the live cell library and a drug droplet from each of the plurality of drug libraries.

S3. The system of any of the system embodiment(s) S1-S2, wherein a merge set is uniquely identifiable by: (i) the unique cell identifier of the particular cell, and (ii) the unique drug identifiers of the drug droplets comprising the merge set.

S4. The system of any of the system embodiment(s) S1-S3, wherein drugs in a merge set are identifiable by the identifiers of the drug droplets comprising the merge set.

S5. The system of any of the system embodiment(s) S1-S4, further constructed and adapted to:
determine, from plurality of merged sets, effectiveness of a set or sets of drugs with respect to one or more criteria; and
identify the drugs in the set or sets of drugs that were the effective with respect to the one or more criteria.

S6. The system of any of the system embodiment(s) S5, wherein the system identifies the set or sets of drugs that were most effective with respect to the one or more criteria.

S7. The system of any of the system embodiment(s) S5-S6, wherein the system identifies one or more drug combinations based on said drug combination's synergistic effect on a cell according to the one or more criteria.

S8. The system of any of the system embodiment(s) S1-S7, wherein the unique drug identifier for a drug in the plurality of drug libraries comprises a unique DNA sequence for the drug, and wherein the unique cell identifier for a cell in said live cell library comprises a unique DNA sequence for the cell, and wherein the system is constructed and adapted to identify the drugs in the set or sets of drugs by:
concatenating the unique DNA sequences in each set; and
sequencing concatenated unique DNA sequences.

S9. The system of any of the system embodiment(s) S5-S8, wherein the one or more criteria are selected from: live versus dead, protein abundance, presence or intensity of cellular proteins, metabolites or metabolite detection, nucleic acid detection, DNA, RNA, and/or other biomarkers.

S10. The system of any of the system embodiment(s) S1-S9, wherein the plurality of junctions combine streams of drug droplets from each of the plurality of drug libraries with a stream of live cells from the live cell library.

S11. The system of any of the system embodiment(s) S1-S10, wherein the plurality of junctions comprise: one or more first junctions for combining a stream of drug droplets from each of the plurality of drug libraries.

S12. The system of any of the system embodiment(s) S11, wherein the one or more first junctions form a stream of drug droplet sets.

S13. The system of any of the system embodiment(s) S1-S12, wherein the plurality of junctions further comprise a second junction combining live cells from said live cell library with said drug droplet sets.

S14. The system of any of the system embodiment(s) S13, wherein the second junction forms a plurality of merged sets, each of said merged sets comprising a particular cell from the live cell library and a drug droplet from each of the plurality of drug libraries.

S15. The system of any of the system embodiment(s) S2-S14, wherein each merge set is uniquely identifiable by the unique cell identifier of the particular cell and the identifiers of the drug droplets.

S16. The system of any of the system embodiment(s) S1-S15, wherein the corresponding unique drug identifier for a drug in the drug libraries comprises a unique DNA drug identifier.

S17. The system of any of the system embodiment(s) S1-S16, wherein the corresponding unique cell identifier for a cell in the live cell library comprises a unique DNA cell identifier.

S18. The system of any of the system embodiment(s) S16-S17, wherein the unique DNA drug identifier for a drug in the plurality of drug libraries comprises a unique DNA sequence for the drug.

S19. The system of any of the system embodiment(s) S1-S18, wherein the unique DNA cell identifier for a cell in said live cell library comprises a unique DNA sequence for the cell.

S20. The system of any of the system embodiment(s) S1-S19, wherein drugs in a merge set are identifiable by the unique DNA drug identifiers of the drug droplets comprising the merge set.

S21. The system of any of the system embodiment(s) S1-S20, wherein the plurality of drug libraries consist of two drug libraries.

S22. The system of any of the system embodiment(s) S1-S21, wherein each of the plurality drug libraries contains up to 1,000 drugs, more preferably up to 2,000 drugs, and even more preferably up to 5,000 drugs.

S23. The system of any of the system embodiment(s) S1-S22, wherein the live cell library contains 1 to 100 cell lines.

S24. The system of any of the system embodiment(s) S1-S23, wherein each of said drug libraries produces a stream of drug droplets, said drug droplets corresponding to said drugs in said drug libraries.

S25. The system of any of the system embodiment(s) S1-S23, wherein at least one of said drug libraries produces a stream of heterogenous drug droplets, said drug droplets corresponding to said drugs in said drug libraries.

S26. The system of any of the system embodiment(s), wherein at least one of said drug libraries produces a random stream of heterogenous drug droplets.

S27. The system of any of the system embodiment(s) S1-S26, wherein the unique drug identifier for a drug in the system comprises an optical identifier associated with the drug.

S28. The system of any of the system embodiment(s) S27, wherein the optical identifier comprises a dye.

S29. A system comprising:
a plurality of libraries, at least one of the libraries comprising heterogenous droplets; and
one or more junctions combining droplets from the libraries, wherein each droplet is identifiable within the system.

S30. The system of any of the system embodiment(s) S29, wherein the one or more junctions form a plurality of merged sets of droplets, wherein a merged set has a droplet from each of the plurality of libraries.

S31. The system of any of the system embodiment(s) S29-S30, wherein the plurality of libraries comprise a plurality of drug libraries, at least one comprising multiple drugs, each drug associated with a corresponding unique drug identifier in the system.

S32. The system of any of the system embodiment(s) S29-S31, wherein the plurality of drug libraries consists of two drug libraries.

S33. The system of any of the system embodiment(s) S29-S32, wherein the plurality of libraries comprise a live cell library comprising multiple live cells or live cell lines, each live cell associated with a corresponding unique cell or cell-line identifier in the system.

Below is a list of process (or method) embodiments. Those will be indicated with a letter “P”. Whenever such embodiments are referred to, this will be done by referring to “P” embodiments.

P34. A method comprising:
merging a set of drugs from a plurality of drug libraries with live cells from a live cell library to form corresponding merged sets, wherein at least some of said plurality of drug libraries comprise multiple droplets of multiple drugs, each drug associated with a corresponding unique drug identifier, and wherein the live cell library comprising multiple live cell types, each live cell type associated with a corresponding unique cell identifier; and then
determining, from the cells that responded, effectiveness of a set or sets of drugs with respect to one or more criteria; and then
identifying the drugs in the set or sets of drugs that were effective with respect to the one or more criteria.

P35. The method of the process embodiment(s) P34, wherein said identifying identifies the set or sets of drugs that were most effective with respect to the one or more criteria.

P36. The method of any of the process embodiment(s) P34-P35, wherein the one or more criteria are selected from: live versus dead, protein abundance, presence or intensity of cellular proteins, metabolites or metabolite detection, nucleic acid detection, DNA, RNA, and/or other biomarkers.

P37. The method of any of the process embodiment(s) P34-P36, further comprising incubating said merged sets prior to said determining.

P38. The method of any of the process embodiment(s) P34-P37, further comprising, prior to said determining, partitioning the merged sets based on their cells' responses to the drugs, according to the one or more criteria.

P39. The method of any of the process embodiment(s) P38, wherein said partitioning comprises separating cells that responded to the drugs according to the one or more criteria from cells that did not respond according to the one or more criteria.

P40. The method of any of the process embodiment(s) P34-P39, further comprising determining a matrix of drug combination responses.

P41. The method of any of the process embodiment(s) P40, wherein an entry in the matrix for a particular drug combination corresponds to the particular drug combination's synergistic effect on a cell according to the one or more criteria.

P42. The method of any of the process embodiment(s) P34-P41, wherein the unique drug identifier for a particular drug comprises a unique DNA sequence for the particular drug, and wherein the unique cell identifier for a particular live cell comprises a unique DNA sequence for the particular live cell.

P43. The method of any of the process embodiment(s) P34-P42, wherein said identifying the drugs comprises concatenating the unique DNA sequences in each set.

P44. The method of any of the process embodiment(s) P43, further comprising sequencing concatenated unique DNA sequences.

P45. The method of any of the process embodiment(s) P34-P44, wherein the unique drug identifier for a particular drug comprises a unique optical identifier for the particular drug, and wherein the unique cell identifier for a particular cell comprises a unique optical for the particular cell.

P46. The method of any of the process embodiment(s) P34-P45, wherein each merge set is uniquely identifiable by the unique cell identifier of the cell in the merge set and the identifiers of the drugs in the merge set.

P47. The method of any of the process embodiment(s) P34-P46, further comprising creating said plurality of drug libraries.

P48. The method of any of the process embodiment(s) P34-P47, further comprising creating said live cell library.

P49. The method of any of the process embodiment(s) P34-P48, wherein said plurality of drug libraries consists of two drug libraries.

P50. The method of any of the process embodiment(s) P34-P49, wherein each of said drug libraries contains up to 1,000 drugs, more preferably up to 2,000 drugs, and even more preferably up to 5,000 drugs.

P51. The method of any of the process embodiment(s) P34-P50, wherein each of said drug libraries produces a stream of drug droplets, said drug droplets corresponding to said drugs of said drug libraries.

P52. A method, in a system comprising a plurality of libraries; and one or more junctions, the method comprising: combining droplets from the libraries, wherein each droplet is identifiable within the system.

P53. The method of any of the previous process embodiments P34-P52 on the system of any of the system embodiments S1-S33.

S54. The system of any of the system embodiments S1-S33, carrying out the process or method of any of the process embodiments P34-P52.

The above features along with additional details of the invention, are described further in the examples herein, which are intended to further illustrate the invention but are not intended to limit its scope in any way.

### BRIEF DESCRIPTION OF THE DRAWINGS

Objects, features, and characteristics of the present invention, as well as the methods of operation and functions of the related elements of structure, and the combination of parts and economies of manufacture, will become more apparent upon consideration of the following description and the appended claims with reference to the accompanying drawings, all of which form a part of this specification.

FIGS. 1A-1B and 2A-2B depict aspects of frameworks for screening drug combinations according to exemplary embodiments hereof;

FIG. 3 shows aspects of an exemplary 2-dimensional matrix showing the synergistic effect on cell death of various drug pairs;

FIG. 4A depicts aspects of a drug library according to exemplary embodiments hereof;

FIG. 4B depicts aspects of a cell library according to exemplary embodiments hereof;

FIG. 4C is a photograph of a drug or cell library according to exemplary embodiments hereof;

FIGS. 4D-4G depict aspects of droplet stream combining/merging mechanisms or junctions according to exemplary embodiments hereof;

FIG. 5 depicts aspects of combining libraries according to exemplary embodiments hereof;

FIG. 6 depicts aspects of a framework for screening drug combination pairs according to exemplary embodiments hereof;

FIGS. 7A-7C depict aspects of merged drug/cell sets according to exemplary embodiments hereof;

FIG. 8 is a flowchart of exemplary operation of aspects of a framework of FIGS. 1, 2, and/or 6, according to exemplary embodiments hereof.

### DETAILED DESCRIPTION OF EXEMPLARY EMBODIMENTS

As used herein, the following terms have the following meanings unless specifically stated otherwise:

The term “mechanism,” as used herein, refers to any device(s), process(es), service(s), or combination thereof. A mechanism may be mechanical or electrical or a combination thereof. A mechanism may be integrated into a single device, or it may be distributed over multiple devices. The various components of a mechanism may be co-located or distributed. The mechanism may be formed from other mechanisms. In general, as used herein, the term “mechanism” may thus be considered shorthand for the term device(s) and/or process(es) and/or service(s).

### DESCRIPTION

Identifying Drugs and Cells

Aspects hereof require identifying individual drugs (and cells) that make up a combination of two or more drugs with at least one cell. To this end, drugs and cells within the system may be associated with unique identifiers (i.e., with identifiers or identities that are unique within the system).

Unless stated otherwise or apparent from the context, drugs in a combination or combinations of drugs are identifiable using techniques such as those described here.

Cells may be uniquely identified within the system using DNA identifiers such as so-called DNA barcodes (unique DNA sequences), optical identifiers (e.g., dyes or the like), or any other means.

For example, according to exemplary embodiments hereof, cells may be linked to DNA identifiers by embedding the cells in a gel matrix, and then linking the same gel matrix to DNA identifiers. Cells can be embedded in a gel matrix, e.g., as described in U.S. published patent application number 20190160445 A1, the entire contents of which are hereby fully incorporated herein by reference for all purposes.

The gel matrix may be functionalized to enable binding to DNA cell identifiers and to DNA drug identifiers. This binding may be enabled by conjugating DNA molecules to the gel matrix to enable capture of complementary DNA cell identifiers and drug identifiers. In other exemplary embodiments, DNA identifiers may be linked to the gel matrix by chemistry. A number of methods are well known in the art such as the conjugation of streptavidin proteins to the gel matrix to enable capture of biotin labeled drug DNA identifiers and cell DNA identifiers. Alternatively, DNA identifiers may also be directly linked to live cells by a variety of methods known in the art. For example, streptavidin conjugated antibodies may be bound directly to the cell surface with no requirement for a gel matrix, and biotin labeled DNA identifiers may be captured directly onto this bound streptavidin.

One or more first junctions may be used for combining a stream of drug droplets from each of a plurality of drug libraries to form a stream of drug droplet sets where each drug type in each of the plurality of drug libraries is mixed in solution with a drug-DNA identifier. In some cases, the stream of drug droplet sets may be further combined with a library of cells, where each cell may be embedded in a gel matrix, and where each cell type in the library may be mixed in solution with a cell-DNA identifier, to form a stream of drug and cell droplet sets. In some exemplary embodiments, the gel matrix in which cells are embedded may be chemically functionalized to bind to drug-DNA identifiers and to cell-DNA identifiers. In some exemplary embodiments, each of the cell and drug droplet sets may then be merged to form one large droplet where the drugs and cells are combined into a cell-drug-combination droplets.

The cell-drug combination droplets which contain cells embedded in a gel matrix, drug-DNA identifiers, and cell-DNA identifiers, may then be allowed to incubate. During this incubation period, the cell-DNA identifiers and the drug-DNA identifiers bind to the gel matrix.

In some cases, cells embedded in a gel matrix containing cell-DNA identifiers and drug-DNA identifiers may then be collected from the cell-drug-combination droplets and are subjected to a drug response assay. For example, the drug response assay may be a fluorescent assay such as a calcein AM assay for live cells, an ethidium homodimer assay for dead cells, a Hoechst assay for cell nuclei, an apoptosis fluorescent assay, or any other fluorescent assay for cellular pathway activation, protein expression, metabolite presence, or other. In some exemplary embodiments, the fluorescently labeled, gel-embedded cells are then sorted using flow cytometry (FACS) by drug effect. For example, gel-embedded cells may be FACS sorted into live and dead populations or other cell effects. After sorting, cell-DNA identifiers and drug-DNA identifiers may be combined (e.g., concatenated) so that they form a single molecule.

Cell-DNA identifiers and drug-DNA identifiers are designed to enable identification of all possible three-drug combinations as described in U.S. published patent application number 20170029813 A1, the entire contents of which are hereby fully incorporated herein by reference for all purposes. For this purpose, three barcode sets may be designed (sets A, B, and C), where each barcode contains a drug-ID unique to each drug in the set, and flanking universal tags, common to all tags in the set. In addition, the universal tags can be designed such that the A, B, and C sets, when PCR amplified together, form a single strand of DNA, unique to each A-B-C drug combination.

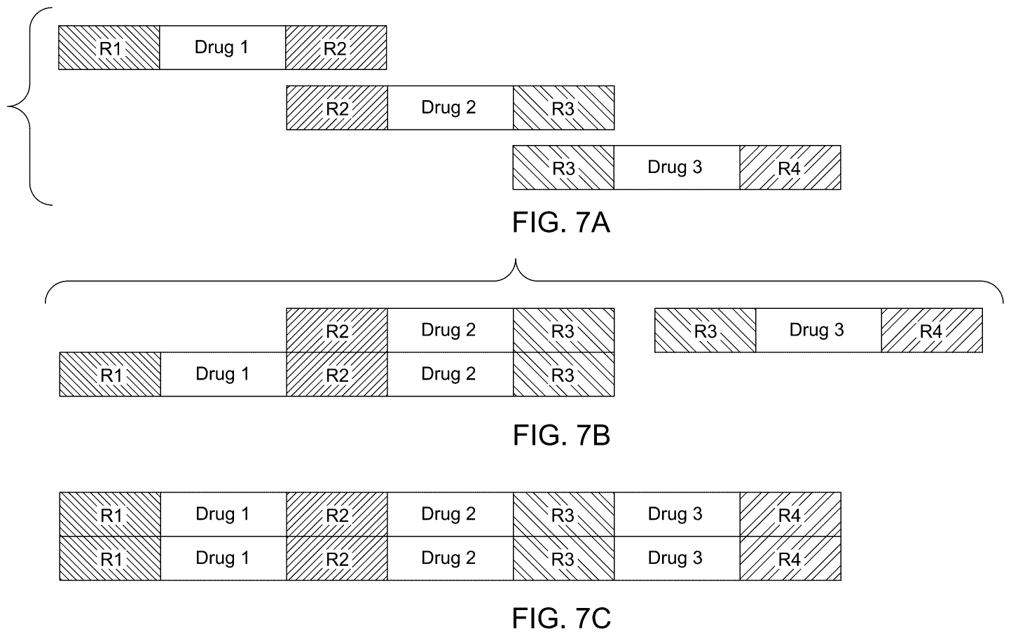

FIGS. 7A-7C show aspects of an exemplary barcode design scheme (a barcode oligonucleotide design for a 3-drug combination screen). In this exemplary barcode design, three (3) oligonucleotides (A, B, and C) are used for each drug (in this example, drug ID1). The shaded regions (colors) represent homologous sequence regions for PCR amplification of a combined ABC PCR product. FIG. 7B shows PCR products after the first PCR cycle. In this example, the three drug ID's are ID1, ID7, and ID13. FIG. 7C shows the combined PCR product is created starting with PCR cycle 2. Three barcode IDs identify the three combined drugs.

In some exemplary embodiments, gel-embedded cells may be mixed with PCR reagents and are emulsified at a low density of gels to emulsion droplets. For example at a concentration of 5% to 30% gel-volume to droplets-volume concentration, and then thermally-cycled for PCR amplification for the purpose of concatenation of the drop-DNA identifiers and the cell-DNA identifiers as described in US 20170029813 A1.

In some exemplary embodiments concatenated drug-DNA identifiers and cell-DNA identifiers are then DNA sequenced to established which sets of drugs and cells are enriched in each of the sorted drug responses to suggest which drug combinations are effective for each cell type tested.

### DESCRIPTION

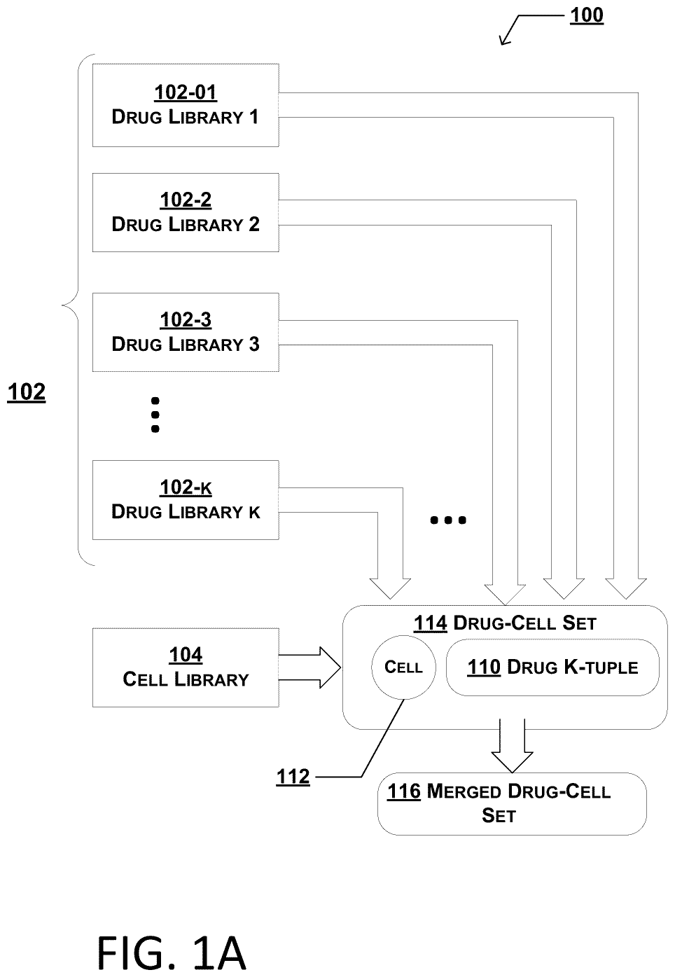

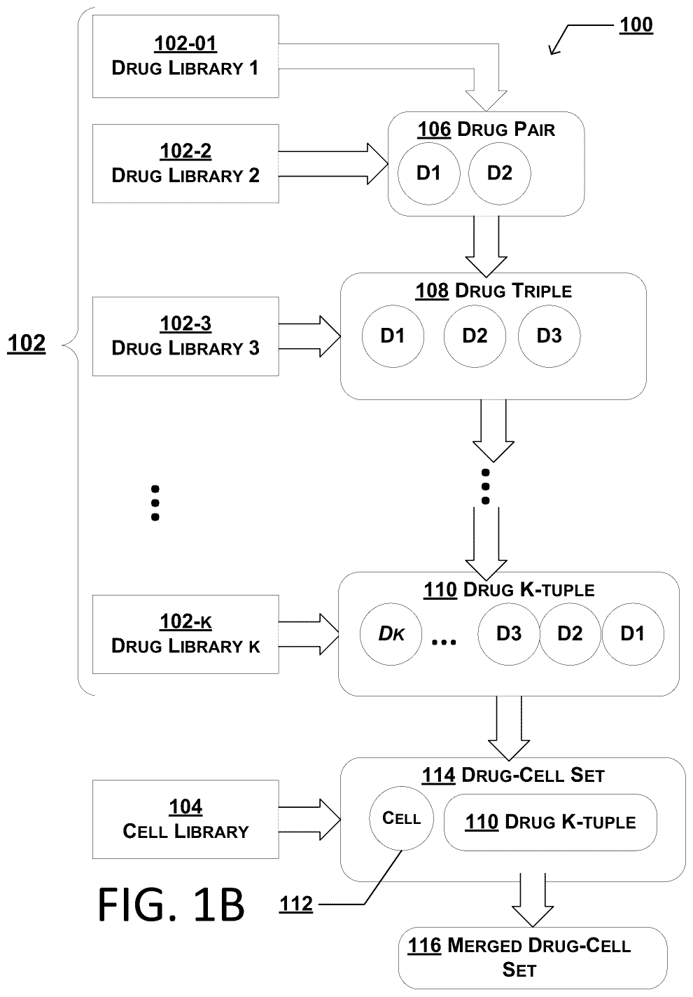

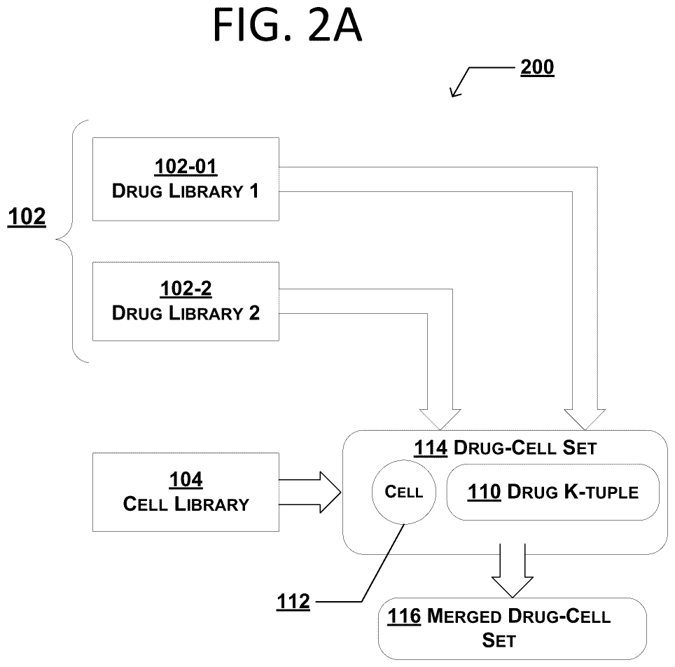

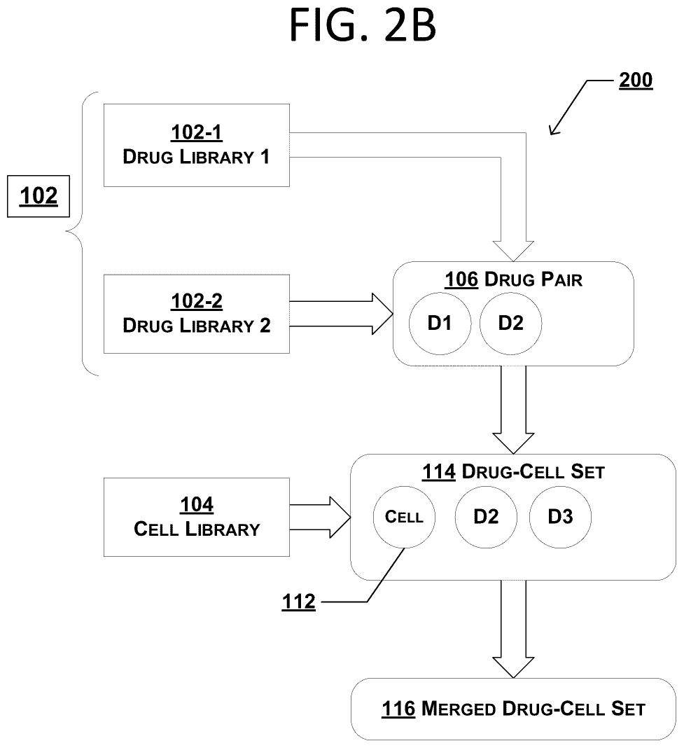

With reference now to FIGS. 1A-1B, a framework or system 100 for screening drug combinations includes k drug libraries 102-1, 102-2, 102-3 . . . 102-k for k≥2 (individually and collectively drug library(ies) 102) and at least one cell library 104. FIGS. 2A-2B show example frameworks or systems 200 with k=2, i.e., with only two drug libraries (102-1, 102-2).

The j-th drug library 102 may have jₙ drugs, for some jₙ≥1. Those of skill in the art will understand, upon reading this description, that the value of jₙ (number of drugs in the j-th drug library) may be any integer value, and the system is not limited by the value of jₙ (i.e., by the number of drugs in a drug library). In some current exemplary implementations jₙ is up to 1,000, although higher numbers are contemplated. The number of drugs in a drug library may be in the range 1 to 50, more preferably in the range 1 to 96, even more preferably in the range 1 to 500, and even more preferably in the range 1 to 1,000, and even more preferably in the range 1 to 2,000, and even more preferably in the range 1 to 5,000. As will also be appreciated, upon further reading of this description, while jₙ may equal 1 for some of the drug libraries, at least one drug library 102 has jₙ>1.

The various drug libraries 102 need not all have the same drugs or the same number of drugs. Thus, in some systems, one or more of the libraries may have different numbers of drugs and the drugs in each library may differ.

In each drug library 102, each drug is uniquely identifiable in a manner such that the identity of the drug can be determined from an appropriate amount of the drug (e.g., a droplet or greater amount), even when mixed with other drugs or things in the system. For example, each drug may be identified by a unique identifier (i.e., with an identifier that is unique within the system), and the drug may be associated with its unique identifier in a manner that allows determination of the drug's unique identifier from an amount of the drug. Various ways of associating a drug's unique identifier with the drug are described in greater detail herein. The amount of a drug required to identify the drug may depend on the technique(s) used to associate the unique identifier with the drug.

Thus, given an amount of a particular drug (e.g., a droplet), it is possible to determine that drug's unique identifier in the system and thereby to identify the drug.

The cells (or cell library) 104 is a library of live cells, each cell also having a unique cell identifier within the system. Thus, given a particular cell, it is possible to determine that cells unique cell identifier and thereby identify the cell. The manner(s) of associating unique cell identifiers with cells is (are) described in greater detail below.

The system 100 operates by combining a drug from each drug library 102 (of the k drug libraries) with a cell from the cell library. This process is repeated for multiple drugs from the k drug libraries 102 with cells from the cell library, thereby forming multiple combinations of drugs with a cell.

For example, as shown in FIG. 1B, a pair of drugs {D1, D2} 106 is formed, with D1 being a drug from drug library #1 102-1 and D2 being a drug from drug library #2 102-2. The pair of drugs 106 may be in any order and is thus depicted as a set.

The drug pair 106 is combined with a drug D3 from drug library #3 to form a three drug set 108 {D1, D2, D3}. This process is repeated for each of the remaining drug libraries 102, to form the k-drug set 110 {D1, D2, . . . Dk}, where drug Dj is a drug from the j-th drug library, j=1 . . . k.

As should be appreciated, since the drug libraries 102 may contain the same drugs, the drugs in the k-drug set 110 may not be unique. That is, it is possible that at least two of the drugs are the same. It is also possible that the same k-drug set 110 (i.e., a set with the same combination of k drugs) may have been previously formed.

The k-drug set 110 is combined with a live cell 112 from the cell library 104 to form a drug-cell set 114 which may be merged to form a merged drug-cell set 116.

Although this process is shown serially in FIG. 1B, the k-drug set 110 may be formed in any order, including in serial or parallel, to obtain a drug from each of the k drug libraries 102. In addition, the live cell 112 may be combined with some of the drugs in the set before the rest of the drugs are added. The general approach, without any ordering shown or implied, is depicted in FIG. 1A.

The above process, described for a particular live cell 112 and k-drug set 110, is repeated for multiple live cells and k-drug sets, producing multiple merged drug-cell sets. The merged drug-cell sets are then processed, as described below.

As will also be appreciated (and explained below), over time, the system may produce most or all combinations of all drugs in the drug libraries 102. That is, over time, each distinct drug in the j-th drug library will combine with each distinct drug in each of the other drug libraries.

The merged set of drugs with live cells are incubated for certain period (e.g., 1 to 7 days), after which the cells may be categorized based on their reactions or responses to the drugs with respect to one or more criteria. The criteria may be selected from: live versus dead, protein abundance (presence or intensity of cellular proteins), metabolites or metabolite detection, nucleic acid detection, DNA, RNA, and/or other biomarkers. Thus, for example, the sets (cell plus drug combinations) may be separated into those that lived are separated from those that died.

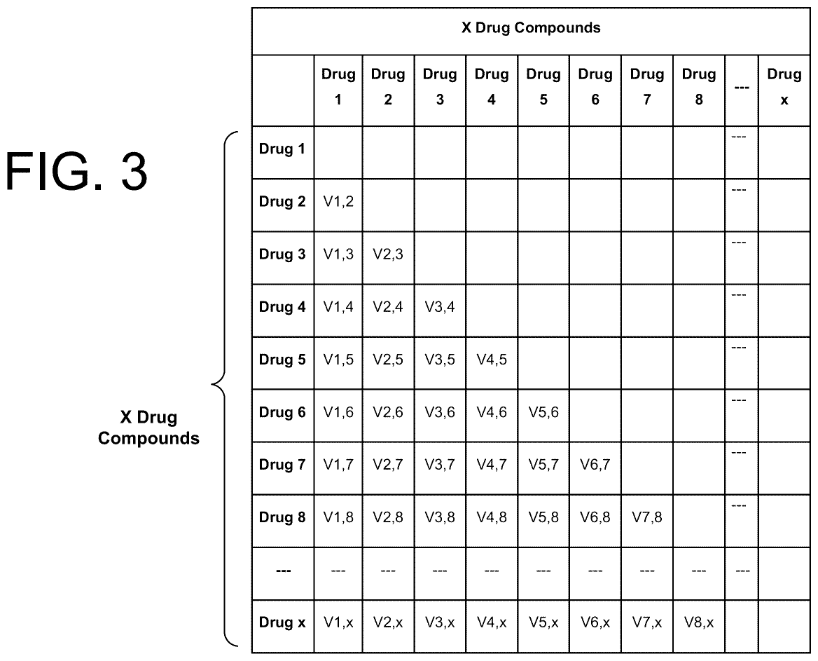

The system then determines which drug sets (i.e., which drug combinations) were most effective (by some measure(s) of effectiveness, e.g., which were most lethal). For example, for a system with two drug libraries (k=2, FIGS. 2A-2B), the system may determine which drug pairs were most effective (e.g., most lethal) and may track this information in a data structure such as a table (e.g., as shown in FIG. 3).

These drug sets may then be sorted by response, and then for the sorted population, the various drugs and cell(s) are identified.

The drugs may be identified using the unique identifiers that were associated with each drug. Thus, e.g., when DNA barcodes are used to encode the unique identifiers, these DNA barcodes may be determined (e.g., as described below).

For a system with two drug libraries, the first drug library having J₁ drugs, and the second drug library having J₂ drugs, a two-dimensional matrix of drug set (drug pair) responses (with size J₁×J₂) may be created, where each matrix entry corresponds to a particular drug pair's synergistic effect on cells (e.g., on cell death, etc.), where a drug pair contains a drug from the first drug library and a drug from the second drug library.

As should be appreciated, the drug pair (A, B) may be treated as equivalent to the drug pair (B, A), and so if the drug libraries are not fully distinct (i.e., if they include some of the same drugs), the duplicates may be consolidated in the matrix.

More generally, for a system with k drug libraries, k≥2, the j-th drug library having Jₙ drugs, a k-dimensional matrix of drug set responses (with size J₁×J₂× . . . Jₖ) may be created, where each matrix entry corresponds to a particular k-drug set's synergistic effect on cells (e.g., on cell death).

A drug set's synergistic effect on a cell, for one or more criteria, may produce a score based on the success of that drug set (or drug combination) on the cell with respect to those one or more criteria. The entry in the matrix for a particular drug set may then be or correspond to that drug set's score.

As should be appreciated, the system may determine different scores (and have different matrices) for different criteria. For example, a system may consider m criteria, m≥2, in which case the system may have m matrices, one for the scores for each of the criteria. Alternatively, some or all of the scores for the different criteria for particular drug set (drug combination) may be combined in a single matrix (e.g., as a weight function of the individual scores).

### Exemplary Implementations

An exemplary implementation of the framework or system 200 (of FIG. 2, with k=2, and thus with two drug libraries (102-1, 102-2)) is described with reference to the drawings.

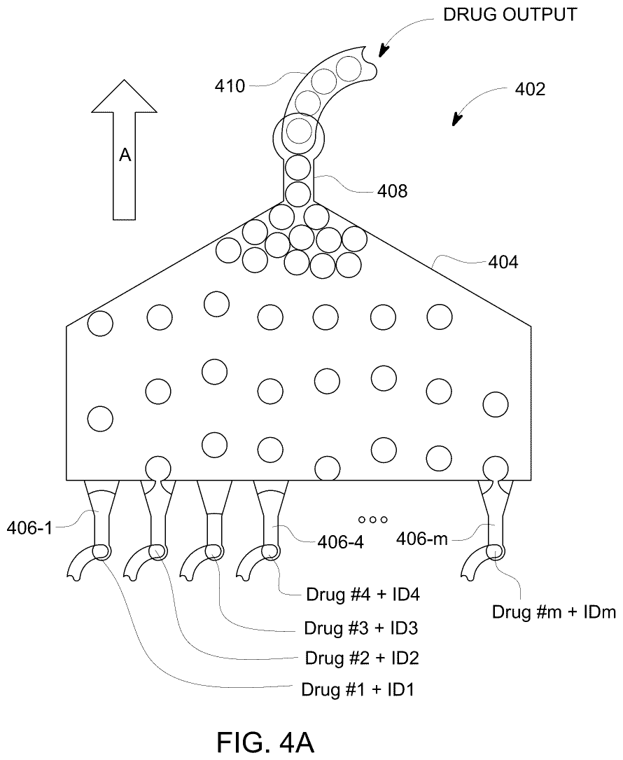

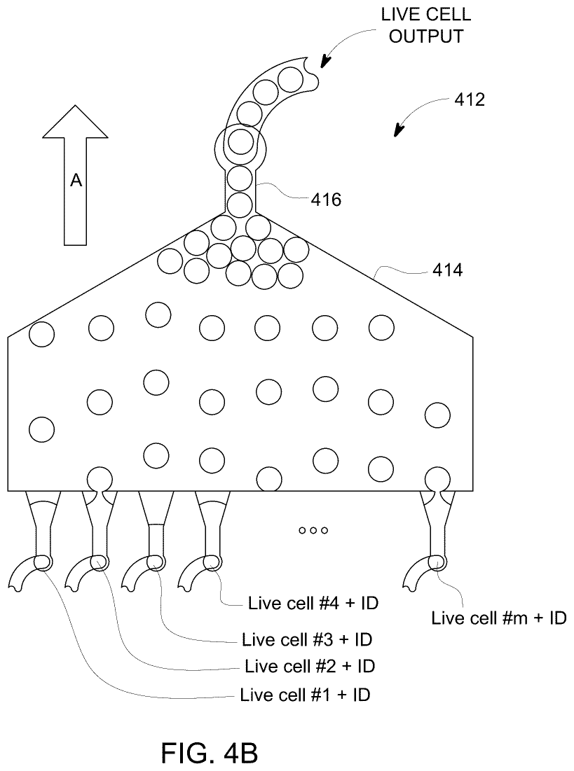

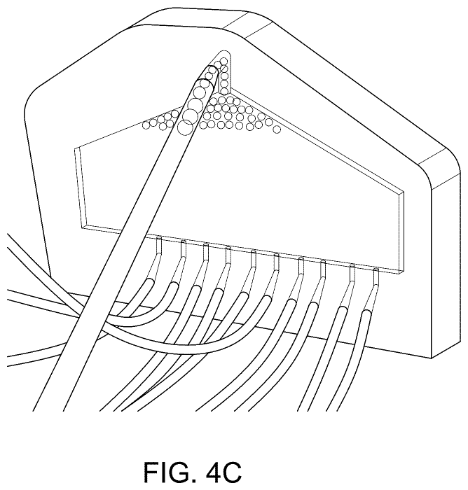

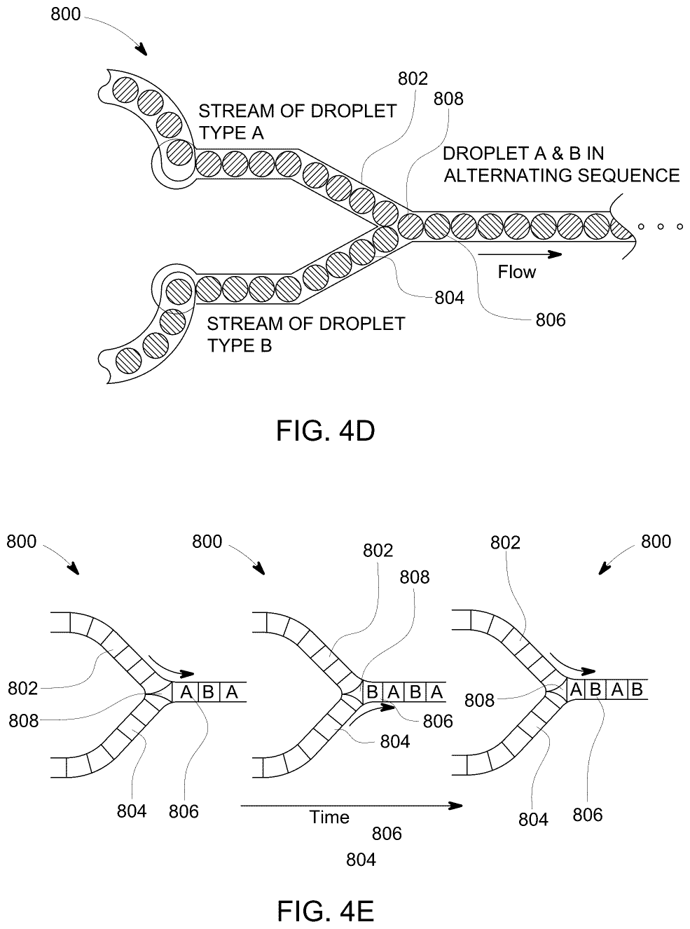

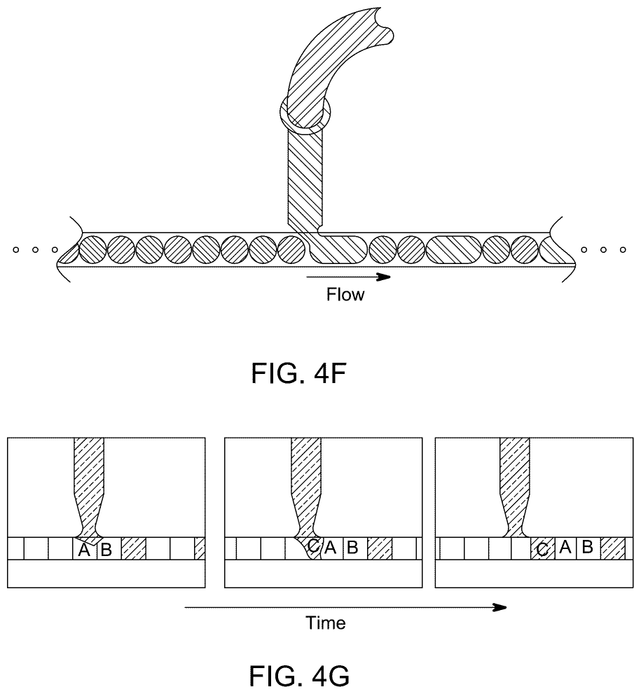

FIG. 4A depicts aspects of a drug library 402 according to exemplary embodiments hereof. Drug library 402 may correspond to drug libraries 102 in FIGS. 1A-1B and 2A-2B.

The drug library 402 comprises m drug sources (labeled “Drug #1+ID, for i=1 . . . m). Each drug source i produces a continuous stream of droplets of the corresponding drug i. Each drug and, therefore, each droplet is identifiable within the system by a unique identifier uniquely identifying drug i within the system. Thus, given a droplet of any drug in the system, it is possible to identify that drug. For example, in cases where each drug is associated with a corresponding DNA barcode (encoding that drug's unique identifier), it is possible to identify each drug droplet from its associated DNA barcode. In the notation “Drug #1+ID” the plus sign (“+”) does not imply or impose any structural or implementation restriction on the system and is a general notation used to show that the drug i is identifiable within the system by being associated in some way with a corresponding identifier that is unique within the system.

A drug droplet that has a corresponding unique identifier (e.g., in the form of a DNA barcode) may be referred to as a tagged drug droplet. In the description below, a tagged drug droplet may be rereferred to as a drug droplet when it is clear from the context that the drug droplet is tagged. Those of skill in the art will understand, upon reading this description, that the word “tagged” does not mean physically bound to unless so stated.

The drug droplets from a drug source enter the bottom of an accumulation area or buffer chamber 404 that contains a liquid (e.g., an oil such as mineral oil, silicone oil, fluorinated oil, or the like), and float vertically (in the direction of the arrow “A” in the drawing). The drug droplets may enter the accumulation area or buffer chamber 404 through a plurality (m) of nozzles 406-1 . . . 406-m (individually and collectively nozzle(s) 406), each of which produces a single drug droplet. The drug droplets accumulate at the top of the buffer chamber 404 and exit through an outlet tube 408 that may be connected, e.g., to another tube 410.

The liquid in the buffer chamber 404 may be an oil (e.g., mineral oil, silicone oil, fluorinated oil, or the like), and is chosen to allow the drug droplets in the buffer chamber 404 to float upward (in the direction of the arrow “A”) toward the outlet tube 408.

The outlet tube 408 is sized to allow only one drop to exit the buffer chamber 404 at a time. Droplets (i.e., the identifiable drug droplets) preferably leave the buffer chamber 404 through outlet tube 408 with little or no gaps between them.

The m drug sources may each provide a unique drug (that is, each drug #i may differ from each other drug #j, for i≠j), or some of the drug sources may be the same.

As should be appreciated, the probability of a particular drug droplet in the buffer chamber 404 is a particular drug j depends, at least in part, on the rate of input of drug j into the buffer chamber 404 relative to the other drugs. Similarly, the rate of a particular drug droplet that exits the drug library 402 (via the outlet tube 408) being a particular drug j depends, at least in part, on the rate of input of drug j into the buffer chamber 404 relative to the other drugs in the buffer chamber 404.

As should be appreciated, over time drug droplets from each drug source will exit the drug library 402 via its outlet tube 408.

FIG. 4B depicts aspects of a cell library 412 according to exemplary embodiments hereof. Cell library 412 corresponds to cell library 104 in FIGS. 1 and 2.

The cell library 412 has a similar overall structure as the drug library 402 described above with reference to FIG. 4A, with a corresponding oil-filled accumulation/buffer chamber 414, and outlet tube 416, except that instead of drug droplets being input though ports at the bottom of the buffer chamber, live cells (or live cell droplets) are input through the ports.

The liquid in the buffer chamber 414 may be a cell culture media, and is chosen to allow the live cells (or live cell droplets) to float upward (in the direction of the arrow “A”).

The outlet tube 416 is sized to allow only one live cell (or live cell droplet) to exit the buffer chamber 414 at a time. Live cells (or live cell droplets) preferably leave the buffer chamber 414 via the outlet tube 416 with little or no gaps between them.

### Merging Droplet Streams

As described, various droplet streams are arranged into discrete sets, possibly ordered. In addition, ordered discrete sets of droplets may be isolated and combined into unified droplets.

### Background

Emulsion microfluidic assays are often used to combine different fluids, for example, for the performance of biological assays. Combining fluids has been a challenge since the droplets carrying the fluids need to come into contact with one another so they can be fused together, without over-fusing with adjacent droplet sets. As an example, if a fluid droplet carrying a live cell should be combined with a second fluid droplet carrying a unique drug, the challenge is to ensure the cell and drug droplets are merged as a pair but do not merge with any other cells and drugs in adjacent droplets. In addition, combining fluid sets requires droplets to be packed into a microfluidic channel at a specific droplet order for downstream processing. For example, sets of droplets containing assay components may have to be ordered before they are, for example, combined together into a single larger drop. Droplet ordering may also be needed in cases where a barrier is required to be introduced between sets of droplets, such as an air spacer, oil spacer, or an immiscible oil barrier. The system described here is designed in one aspect, to order droplets arriving from various sources into single-file arranged in periodic patterns, and in another aspect to first arrange droplets into period patterns and then merge them.

Approach Description

Aspects and embodiments hereof enable continuous alternation of two or more droplet chains with high efficiency. With reference to FIG. 4D, an exemplary device 800 may include two or more inlet channels 802, 804 converging into a single channel 806 at a junction 808 downstream (FIGS. 4D and 4E).

FIG. 4D schematically shows aspects of a continuous ordering and alternation of two fluidic droplet chains into periodic pattern. FIG. 4E shows corresponding time-lapse images showing that two types of droplet chains alternate at the junction of the channel.

The width and height of the channels are designed to be the same or smaller than the diameter of the droplets. This ensures that moving droplets are confined by the channels and aligned into a single file. In this design, two or more types of droplets are emulsified separately prior to entering the device. The droplets are first pooled to form a chain and then introduced into inlet channels. When arriving at the junction, the droplets in the two chains enter the channel alternatively forming a single chain with the alternate pattern.

### Example

In one exemplary implementation, we generated two or more streams of droplets (A and B or more) with a size of 25 microns to 2000 microns or larger. We introduce them into the device with the cross-sectional dimension smaller than the drop's diameter. For example, if the droplet's diameter is 900 microns the channel diameter could be 600 microns×700 microns or other dimensions, that would cause the droplet to be compressed inside the channel. As FIG. 4E shows, droplets in chains A and B arrive at the junction and dovetail into the downstream channel. Our device alternates droplets with high efficiency for example, ˜96% (efficiency=# of correct alternation/# of total events) (n>200).

The complexity of periodic patterns may be increased by introducing additional droplets of the same or different types downstream in a controlled frequency. In one embodiment, a fluid stream is fed via an inlet channel, into a junction in which a single file ordered droplet-set is flowing. Droplets are then generated from the fluid stream, as they are periodical, at set intervals, inserted into the ordered set between everyone, two, three, four, five, six, seven, eight, nine, ten, or more droplets. In another embodiment, droplets are first generated and then fed via an inlet channel to a junction where they are periodically inserted into the ordered set between everyone, two, three, or up to ten or more droplets. The size of the inserted droplets and the frequency of insertion can be easily tuned by changing the flow rate of the inlet and drop-set channel flow. In one embodiment, the additional droplets are immiscible to other droplets in the set and to the continuous phase, thus the insertion of the additional droplets creates a droplet of a different type. In another embodiment, the additional droplet can be introduced so that the droplet sets, and the continuous phase can be surrounded by the additional droplet.

FIG. 4F schematically depicts aspects of insertion of droplets in a pre-ordered droplet chain to generate more complicated periodic pattern. FIG. 4G depicts corresponding time-lapse images showing the in situ generation and insertion of a third droplet between every two droplets in the pre-ordered horizontal droplet chain.

As should be appreciated, this approach enables fluidic ordering and arrangement of droplets from different sources into periodic patterns.

Alternation of two-droplet chains and periodic insertion of additional droplets are depicted. However, those of skill in the art will understand, upon reading this description, that alternation of multiple droplet chains and insertion of several droplets downstream can be achieved by designing additional inlet channels and injection channels.

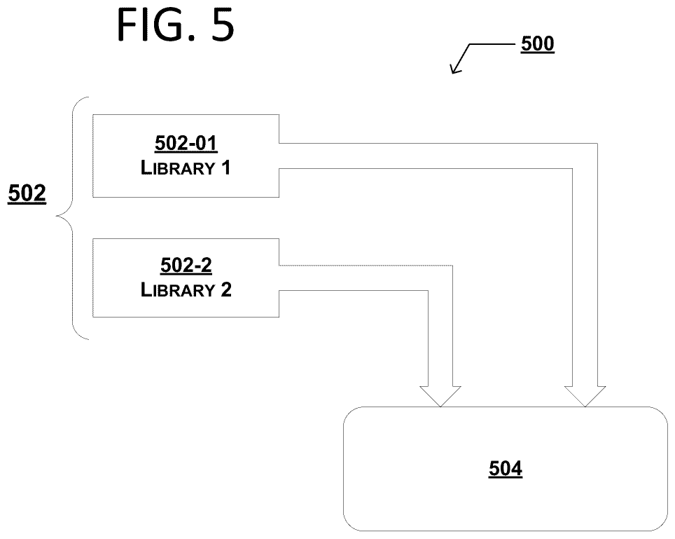

The above examples (e.g., FIGS. 4D-4E) show aspects of specific implementations of combining droplets from libraries. Those of skill in the art will understand, upon reading this description, that, in general, droplets from any two libraries may be combined. For example, as shown in FIG. 5, in a system 500, two libraries 502-1 and 502-2 (collectively, libraries 502) may be combined to form pairs of droplets 504 from the libraries 502. The system 500 may combine the droplets from the two libraries 502 in all 2-way combinations. The libraries 502 may include drug libraries and/or cell libraries. As should be appreciated, the droplets from the two libraries 502 may be merged using the exemplary combining/merging mechanisms and/or junctions described above with reference to FIGS. 4D-4E.

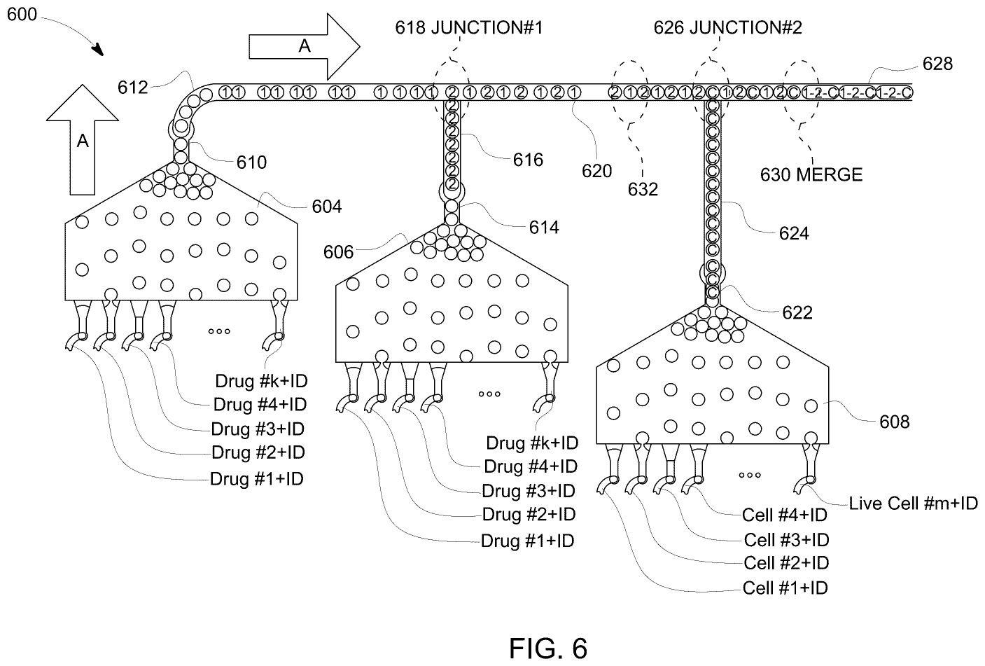

FIG. 6 depicts aspects of a system/framework 600 for screening drug combination pairs according to exemplary embodiments hereof. The framework 600 includes two drug libraries 604, 606 which may be implemented using the drug library discussed above with respect to FIG. 4A. The framework 600 may also include a live cell library 608, which may be implemented using the live cell library 408 discussed above with respect to FIG. 4B. Framework 600 corresponds to the framework 200 in FIGS. 2A-2B. The drug libraries 604, 606 correspond the drug libraries 102-1, 102-2 in FIGS. 2A-2B, and the cell library 608 corresponds to the live cell library 104 in FIGS. 2A-2B.

In operation, drug droplets (denoted “1” or “D1”) exit the drug library #1 (604) one at a time, through an exit port 610 and into a tube 612, forming a first stream of drug droplets from the first drug library 604. Similarly, drug droplets (denoted “2” or “D2”) exit the drug library #2 (606) one at a time, through an exit port 614, forming a second stream of drug droplets in a tube 616.

The first stream of drug droplets is merged with the second stream of drug droplets at a first merge junction 618, which may be a junction such as shown in FIGS. 4D-4E or 4F-4G. The first merge junction 618 forms an alternating series of droplets from the first stream and the second stream, thus creating a stream of drug droplet pairs (each pair having a drug droplet from first drug library 604 and a drug droplet from the second drug library 606). The stream of droplet pairs may be carried in a tube (or tube portion) 620.

Live cells (denoted “C” in the drawing) leave the cell library 608 through the exit port 622 as a stream of cells. The stream may be transported by a tube 624 that connects with the tube (or tube portion) 620 at a second merge junction 626. At the second merge junction 626, the stream of drug pairs (carried in the tube 620) is merged with the stream of live cells (carried in the tube 624), forming a stream of triples, each comprising a drug pair and a live cell. Thus the pair of drugs <D1, D2> is merged at the second merge junction 626 with a live cell C to form a triple <C, D1, D2>. The second merge junction may be a junction such as shown in FIGS. 4D-4E or 4F-4G. The triples (<C, D1, D2>) may be transported via a tube or tube portion 628 to a merge junction 630, where the droplets may be merged. In presently preferred implementations, the C-D1-D2-C-D1-D2-C-D1-D2 packed droplets are first spaced out with oil or air. Then the D1 and D2 droplets catch up the corresponding cell C. The end result is C-D1-D2, oil space, C-D1-D2, oil space, repeated. Then each set is merged.

With reference to FIGS. 6 and 7A-7B, output of the merge junction 630 is series of merged drugs/cell (denoted “1-2-C” in the drawing, corresponding to drug #1 from drug library #1, drug #2 from drug library #2, and a live cell from the cell library).

In some case (not shown), the drug pairs may be merged into a single droplet prior to the second merge junction 626, e.g., at location 632.

As described, the merged cell and drugs exiting the merge junction 630 may then be incubated (for an appropriate period, e.g., 1-7 days). Then the cells are then categorized and sorted based on one or more criteria (e.g., cells that lived vs. cells that died, presence or intensity of cellular proteins, metabolites, DNA, RNA, or other biomarkers). For example, the cells may be categorized based on a measure of lethality, such that those that lived are separated from those that died and the drug pairs that were most lethal may be tabulated and sorted. The drugs (and cells) in the effective combinations are then identified using whatever technique was used to associate the identifiers with the drugs and cells (e.g., using their DNA barcodes or the like).

The identification of the drugs and cell may use, e.g., the concatenated DNA barcode labels shown and discussed with reference to FIGS. 7A-7C.

For each sorted population, the DNA barcodes are extracted, amplified, and sequenced. Then a matrix of drug pair responses is produced (each matrix square corresponds to the drug pair's synergistic effect on the cell, e.g., cell death, etc.).

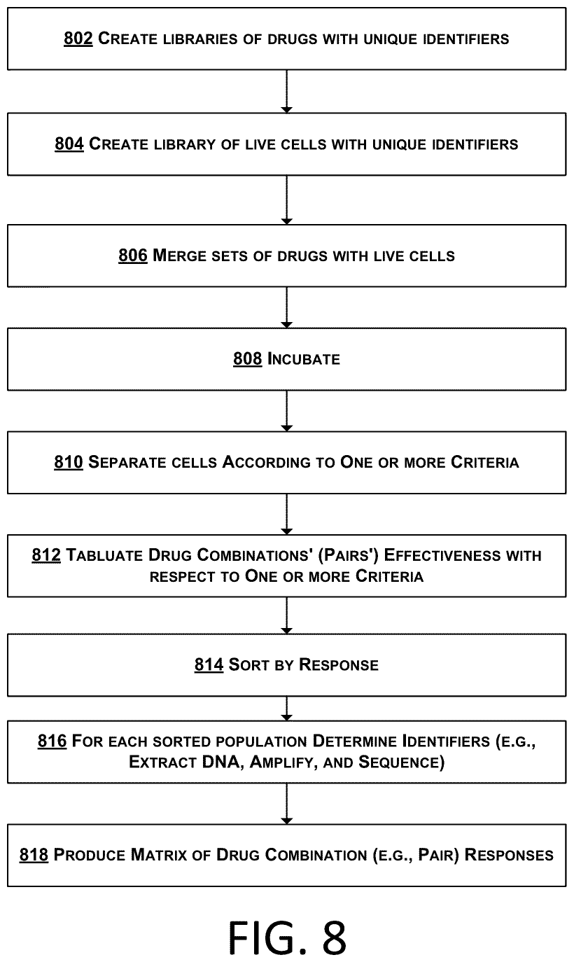

Exemplary operation of aspects of the system may be described with reference to the flowchart in FIG. 8.
Create (or provide) libraries of drugs with unique identifiers (e.g., DNA identifiers such as DNA barcodes or the like) (at 802)
Create (or provide) library of live cells with unique identifiers (e.g., using DNA identifiers such as DNA barcodes or the like) (at 804).
Merge sets of drugs with live cells (at 806).
Incubate the merged sets (e.g., 1 to 7 days) (at 808).
Separate cells based on one or more test criteria (e.g., those that lived from those that died, etc.) (at 810).
Tabulate which drug combinations (e.g., drug pairs when there are two drug libraries) based on their effectiveness with respect to the one or more criteria (e.g., most lethal) (at 812).
Sort by response (at 814).
For each sorted population, determine unique identifiers (e.g., for DNA identifiers: extract DNA, amplify, and sequence) (at 816).
Produce matrix of drug combination (drug pair) responses (each matrix entry corresponds to the drug combination's (i.e., drug pair's) synergistic effect on the cell, based, e.g., on the one or more criteria being evaluated, (e.g., cell death)) at 818.

Once synergistic drug combinations are discovered, those combinations may be further tested to validate or confirm the discovery and then used for clinical trials.

Those of skill in the art will understand, upon reading this description, that drug combinations may be evaluated with respect to the one or more criteria with different degrees or amounts of effectiveness.

### Discussion

Thus is described an ultra-high throughput microfluidic droplet platform which screens matrices of novel drug combinations against libraries of live cells.

It is assumed that over time, all pair-wise combinations of the drugs will be combined with live cells. As should be appreciated, the likelihood of creating all possible combinations is a function of the number of drops generated from each of the k drug library elements. This likelihood may be assessed, for example, by simulation of random pairings across any given library size. So, for example, in a library of 96 drugs, 96² (96×96=9,216) pairings are created. If only 96² drops are created, 50% of the pairs will be combined at least once on average. However, if four times that number of drops is created (4×96²=36,864) 94% of the pairs will be combined at least once.

Since approaches and systems disclosed herein radically reduce the cost of combination screening, increase the speed of testing, and consumes a fraction of reagents required by traditional means. Accordingly, these approaches enable extensive libraries of drugs to be tested. Without imposing selection bias based on cost or speed, embodiments enable vast screens to be evaluated objectively. The new approach holds the promise that novel drug combinations may be identified and pursued for clinical benefit.

### CONCLUSION

Where a process is described herein, those of ordinary skill in the art will appreciate that the process may operate without any user intervention. In another embodiment, the process includes some human intervention (e.g., an act is performed by or with the assistance of a human).

As used herein, including in the claims, the phrase “at least some” means “one or more” and includes the case of only one. Thus, e.g., the phrase “at least some ABCs” means “one or more ABCs”, and includes the case of only one ABC.

As used herein, including in the claims, term “at least one” should be understood as meaning “one or more”, and therefore includes both embodiments that include one or multiple components. Furthermore, dependent claims that refer to independent claims that describe features with “at least one” have the same meaning, both when the feature is referred to as “the” and “the at least one”.

As used in this description, the term “portion” means some or all. So, for example, “A portion of X” may include some of “X” or all of “X”. In the context of a conversation, the term “portion” means some or all of the conversation.

As used herein, including in the claims, the phrase “using” means “using at least,” and is not exclusive. Thus, e.g., the phrase “using X” means “using at least X.” Unless specifically stated by use of the word “only”, the phrase “using X” does not mean “using only X.”

As used herein, including in the claims, the phrase “based on” means “based in part on” or “based, at least in part, on,” and is not exclusive. Thus, e.g., the phrase “based on factor X” means “based in part on factor X” or “based, at least in part, on factor X.” Unless specifically stated by use of the word “only”, the phrase “based on X” does not mean “based only on X.”

In general, as used herein, including in the claims, unless the word “only” is specifically used in a phrase, it should not be read into that phrase.

As used herein, including in the claims, the phrase “distinct” means “at least partially distinct.” Unless specifically stated, distinct does not mean fully distinct. Thus, e.g., the phrase, “X is distinct from Y” means that “X is at least partially distinct from Y,” and does not mean that “X is fully distinct from Y.” Thus, as used herein, including in the claims, the phrase “X is distinct from Y” means that X differs from Y in at least some way.

It should be appreciated that the words “first,” “second,” and so on, in the description and claims, are used to distinguish or identify, and not to show a serial or numerical limitation. Similarly, letter labels (e.g., “(A)”, “(B)”, “(C)”, and so on, or “(a)”, “(b)”, and so on) and/or numbers (e.g., “(i)”, “(ii)”, and so on) are used to assist in readability and to help distinguish and/or identify, and are not intended to be otherwise limiting or to impose or imply any serial or numerical limitations or orderings. Similarly, words such as “particular,” “specific,” “certain,” and “given,” in the description and claims, if used, are to distinguish or identify, and are not intended to be otherwise limiting.

As used herein, including in the claims, the terms “multiple” and “plurality” mean “two or more,” and include the case of “two.” Thus, e.g., the phrase “multiple ABCs,” means “two or more ABCs,” and includes “two ABCs.” Similarly, e.g., the phrase “multiple PQRs,” means “two or more PQRs,” and includes “two PQRs.”

The present invention also covers the exact terms, features, values, and ranges, etc. in case these terms, features, values and ranges etc. are used in conjunction with terms such as about, around, generally, substantially, essentially, at least etc. (i.e., “about 3” or “approximately 3” shall also cover exactly 3 or “substantially constant” shall also cover exactly constant).

As used herein, including in the claims, singular forms of terms are to be construed as also including the plural form and vice versa, unless the context indicates otherwise. Thus, it should be noted that as used herein, the singular forms “a,” “an,” and “the” include plural references unless the context clearly dictates otherwise.

Throughout the description and claims, the terms “comprise”, “including”, “having”, and “contain” and their variations should be understood as meaning “including but not limited to”, and are not intended to exclude other components unless specifically so stated.

It will be appreciated that variations to the embodiments of the invention can be made while still falling within the scope of the invention. Alternative features serving the same, equivalent, or similar purpose can replace features disclosed in the specification, unless stated otherwise. Thus, unless stated otherwise, each feature disclosed represents one example of a generic series of equivalent or similar features.

The present invention also covers the exact terms, features, values, and ranges, etc. in case these terms, features, values and ranges etc. are used in conjunction with terms such as about, around, generally, substantially, essentially, at least etc. (i.e., “about 3” shall also cover exactly 3 or “substantially constant” shall also cover exactly constant).

Use of exemplary language, such as “for instance”, “such as”, “for example” (“e.g.,”) and the like, is merely intended to better illustrate the invention and does not indicate a limitation on the scope of the invention unless specifically so claimed.

While the invention has been described in connection with what is presently considered to be the most practical and preferred embodiments, it is to be understood that the invention is not to be limited to the disclosed embodiment, but on the contrary, is intended to cover various modifications and equivalent arrangements included within the spirit and scope of the appended claims.

1. A system comprising:
a plurality of drug libraries, at least one comprising multiple droplets of multiple drugs, each drug associated with a corresponding unique drug identifier in the system;
a live cell library comprising multiple live cells or live cell lines, each live cell associated with a corresponding unique cell or cell line identifier in the system; and
a plurality of junctions combining drug droplets from the drug libraries and a live cell from the live cell library,
wherein at least one of said drug libraries produces a random stream of heterogenous drug droplets, said drug droplets corresponding to drugs in said drug libraries.

2. The system of claim 1, wherein the plurality of junctions form a plurality of merged sets, each of said merged sets comprising a particular cell from the live cell library and a drug droplet from each of the plurality of drug libraries.

3. The system of claim 2 wherein at least one of the droplets in a set is larger than the other droplets in the set.

4. The system of claim 2, wherein a merge set is uniquely identifiable by: (i) the unique cell identifier of the particular cell, and (ii) the unique drug identifiers of the drug droplets comprising the merge set.

5. The system of claim 2, wherein drugs in a merge set are identifiable by the identifiers of the drug droplets comprising the merge set.

6. The system of claim 2, further constructed and adapted to:
determine, from the plurality of merged sets, effectiveness of a set or sets of drugs with respect to one or more criteria; and
identify the drugs in the set or sets of drugs that were effective with respect to the one or more criteria.

7. The system of claim 6, wherein the system identifies the set or sets of drugs that were most effective with respect to the one or more criteria.

8. The system of claim 6, wherein the system identifies one or more drug combinations based on said drug combination's synergistic effect on a cell according to the one or more criteria.

9. The system of claim 6, wherein the unique drug identifier for a drug in the plurality of drug libraries comprises a unique DNA sequence for the drug, and wherein the unique cell identifier for a cell in said live cell library comprises a unique DNA sequence for the cell, and wherein the system is constructed and adapted to identify the drugs in the set or sets of drugs by:
concatenating the unique DNA sequences in each set; and
sequencing concatenated unique DNA sequences.

10. The system of claim 6, wherein the one or more criteria are selected from: live versus dead, protein abundance, presence or intensity of cellular proteins, metabolites or metabolite detection, nucleic acid detection, DNA, RNA, and/or other biomarkers.

11. The system of claim 1, wherein the plurality of junctions combine streams of drug droplets from each of the plurality of drug libraries with a stream of live cells from the live cell library.

12. The system of claim 1, wherein the plurality of junctions comprise: one or more first junctions for combining a stream of drug droplets from each of the plurality of drug libraries.

13. The system of claim 12, wherein the one or more first junctions form a stream of drug droplet sets.

14. The system of claim 1, wherein the plurality of junctions further comprise a second junction combining live cells from said live cell library with said drug droplet sets.

15. The system of claim 14, wherein the second junction forms a plurality of merged sets, each of said merged sets comprising one or more of a particular cell type from the live cell library and a drug droplet from each of the plurality of drug libraries.

16. The system of claim 1, wherein a cross-sectional dimension of a junction is smaller than diameters of the droplets.

17. The system of claim 15, wherein each merged set is uniquely identifiable by the unique cell identifier of the particular cell type and the identifiers of the drug droplets.

18. The system of claim 1, wherein the corresponding unique drug identifier for a drug in the drug libraries comprises a unique DNA drug identifier.

19. The system of claim 1, wherein the corresponding unique cell identifier for cell types in the live cell library comprises a unique DNA cell identifier.

20. The system of claim 18, wherein the unique DNA drug identifier for a drug in the plurality of drug libraries comprises a unique DNA sequence for the drug.

21. The system of claim 19, wherein the unique DNA cell identifier for a cell in said live cell library comprises a unique DNA sequence for the cell.

22. The system of claim 18, wherein drugs in a merge set are identifiable by the unique DNA drug identifiers of the drug droplets comprising the merge set.

23. The system of claim 1, wherein the plurality of drug libraries consist of two drug libraries.

24. The system of claim 1, wherein each of the plurality drug libraries contains up to 1,000 drugs, more preferably up to 2,000 drugs, and even more preferably up to 5,000 drugs.

25. The system of claim 1, wherein the live cell library contains up to 100 cell lines.

26. The system of claim 1, wherein each of said drug libraries produces a stream of drug droplets, said drug droplets corresponding to said drugs in said drug libraries.

27. The system of claim 1, wherein the unique drug identifier for a drug in the system comprises an optical identifier associated with the drug.

28. The system of claim 27, wherein the optical identifier comprises a dye.

29. A system comprising:
a plurality of libraries, at least one of the libraries comprising heterogenous droplets; and
one or more junctions combining droplets from the libraries, wherein each droplet is identifiable within the system.

30. The system of claim 29, wherein the one or more junctions form a plurality of merged sets of droplets, wherein a merged set has a droplet from each of the plurality of libraries.

31. The system of claim 29 wherein at least one of the droplets in a set is larger than the other droplets in the set.

32. The system of claim 29, wherein at least one of said libraries produces a random stream of heterogenous drug droplets.

33. The system of claim 29, wherein the plurality of libraries comprise a plurality of drug libraries, at least one comprising multiple droplets of multiple drugs, each drug associated with a corresponding unique drug identifier in the system.

34. The system of claim 33, wherein the plurality of drug libraries consists of two drug libraries.

35. The system of claim 29, wherein the plurality of libraries comprise a live cell library comprising multiple live cells or live cell lines, each live cell associated with a corresponding unique cell or cell line identifier in the system.

36. A method comprising:
merging a set of drugs from a plurality of drug libraries with live cells from a live cell library to form corresponding merged sets, wherein at least some of said plurality of drug libraries comprise multiple droplets of multiple drugs, each drug associated with a corresponding unique drug identifier, and wherein the live cell library comprising multiple live cell types, each live cell type associated with a corresponding unique cell identifier,
wherein at least one of said drug libraries produces a random stream of heterogenous drug droplets, said drug droplets corresponding to drugs in said drug libraries; and then
determining, from cells that responded, effectiveness of a set or sets of drugs with respect to one or more criteria; and then
identifying the drugs in the set or sets of drugs that were effective with respect to the one or more criteria.

37. The method of claim 36 wherein at least one of the droplets in a set is larger than the other droplets in the set.

38. The method of claim 36, wherein said identifying identifies the set or sets of drugs that were most effective with respect to the one or more criteria.

39. The method of claim 36, wherein the one or more criteria are selected from: live versus dead, protein abundance, presence or intensity of cellular proteins, metabolites or metabolite detection, nucleic acid detection, DNA, RNA, and/or other biomarkers.

40. The method of claim 36, further comprising:
incubating said merged sets prior to said determining.

41. The method of claim 36, further comprising, prior to said determining, partitioning the merged sets based on their cells' responses to the drugs, according to the one or more criteria.

42. The method of claim 41, wherein said partitioning comprises:
separating cells that responded to the drugs according to the one or more criteria from cells that did not respond according to the one or more criteria.

43. The method of claim 36, further comprising:
determining a matrix of drug combination responses.

44. The method of claim 43, wherein an entry in the matrix for a particular drug combination corresponds to the particular drug combination's synergistic effect on a cell according to the one or more criteria.

45. The method of claim 36, wherein the unique drug identifier for a particular drug comprises a unique DNA sequence for the particular drug, and wherein the unique cell identifier for a particular live cell comprises a unique DNA sequence for the particular live cell.

46. The method of claim 45, wherein said identifying the drugs comprises:
concatenating the unique DNA sequences in each set.

47. The method of claim 46, further comprising:
sequencing concatenated unique DNA sequences.

48. The method of claim 36, wherein the unique drug identifier for a particular drug comprises a unique optical identifier for the particular drug, and wherein the unique cell identifier for a particular cell comprises a unique optical for the particular cell.

49. The method of claim 36, wherein each merge set is uniquely identifiable by the unique cell identifier of the cell in the merge set and the identifiers of the drugs in the merge set.

50. The method of claim 36, further comprising creating said plurality of drug libraries.

51. The method of claim 36, further comprising creating said live cell library.

52. The method of claim 36, wherein said plurality of drug libraries consists of two drug libraries.

53. The method of claim 36, wherein each of said drug libraries contains 1 to 1,000 drugs, more preferably up to 2,000 drugs, and even more preferably up to 5,000 drugs.

54. The method of claim 36, wherein each of said drug libraries produces a stream of drug droplets, said drug droplets corresponding to said drugs of said drug libraries.

55. A method, in a system comprising a plurality of libraries, at least one of the libraries comprising heterogenous droplets; and one or more junctions, the method comprising: combining droplets from the libraries, wherein each droplet is identifiable within the system.

56. The method of claim 55, wherein the one or more junctions form a plurality of merged sets of droplets, wherein a merged set has a droplet from each of the plurality of libraries.

57. The method of claim 56 wherein at least one of the droplets in a set is larger than the other droplets in the set.

58. The method of claim 55, wherein at least one of said libraries produces a random stream of heterogenous drug droplets.

59. The method of claim 55, wherein a cross-sectional dimension of a junction of said one or more junctions is smaller than diameters of the droplets.

60-61. (canceled)
